(19) United States
(12) Patent Application Publication
(10) Pub. No.: US 2003/0100840 A1
(43) Pub. Date: May 29, 2003

(54) Pulse photometry probe

(75) Inventors: Keiichi Sugiura, Tokyo (JP); Toru Maeda, Tokyo (JP)

Correspondence Address: SUGHRUE MION, PLLC, 2100 PENNSYLVANIA AVENUE, N.W., WASHINGTON, DC, 20037, US

(73) Assignee: NIHON KOHDEN CORPORATION

(21) Appl. No.: 10/303,920

(22) Filed: Nov. 26, 2002

(30) Foreign Application Priority Data
Nov. 28, 2001 (JP) P2001-362515

(51) Int. Cl. A61B 5/02

(52) U.S. Cl. 600/504

(57) ABSTRACT

A pulse photometry probe having the following structure is disclosed. A tape-shaped compressive bandage section has a first surface, a second surface opposing to the first surface, a first through hole and a second through hole each connecting the first surface and the second surface. The bandage section is adapted to be wound around a living tissue such that the first surface is brought into contact with the living tissue. A light emitter emits light toward a living tissue. The light emitter detachably attached on a first position of the second surface such that the light passes through the first through hole. A light receiver receives the light which has been emitted from the light emitter and transmitted through the living tissue. The light receiver detachably attached on a second position of the second surface such that the light passes through the second through hole.

### BACKGROUND OF THE INVENTION

The invention relates to a probe for measuring a pulse wave or a light-absorbing material in the blood according to a pulse photometry method.

A conventional probe to be used with a pulse photometry method is described in, e.g., Japanese Patent Publication No. 2-20252B (U.S. Pat. No. 4,830,014). According to this probe, a light source and an optical sensor are fixedly embedded in a support member to be wrapped around a finger. A lower surface of the support member has an adhesive layer. The adhesive layer is brought into contact with the surface of living tissue. As a result, the light source and the optical sensor are fixedly attached to the living tissue, thereby fixing the probe.

Another example of the conventional probe to be used with the pulse photometry method is described in U.S. Pat. No. 5,919,133. In the example, a light emitter and a light receiver are provided on a surface of a soft member to be brought into contact with living tissue. The probe is wrapped around the living tissue through use of an attachment strap.

When a probe having an adhesive layer provided on a support member such as the former example is wrapped around a finger or a like body part of a premature or neonate having delicate skin, the adhesive layer comes into contact with sebum or a horny layer located in the area where the probe is attached. As a result, an adhesive layer is deteriorated, and the adhesive strength of the adhesive layer becomes weak. Further, attachment of the probe causes local pressure. Further, there is a risk of a horny layer being exfoliated by the adhesive layer.

In the case of a probe such as the latter example, a rigid light emitter and a rigid light receiver come into direct contact with living tissue, thus applying pressure to the living tissue. Further, a yoke is provided between the light emitter and the light receiver, and hence a distance between the light emitter and light receivers cannot be controlled. Hence, the optical axis of the probe cannot be adjusted while living tissue, such as a finger, is held between the light emitter and the light receiver. Further, a compressible member and a housing for the light receiver and light emitters, which differ from each other in terms of flexibility, come into contact with living tissue. Hence, when external pressure is applied to the probe while the probe is wrapped around the living tissue, the pressure becomes less likely to be distributed uniformly.

### SUMMARY OF THE INVENTION

It is therefore an object of the invention to provide a probe which effects distribution of pressure without occurrence of necrosis, reddening, or inflammation, which would otherwise be caused by application of local pressure stemming from attachment of the probe, even when the probe is attached to a patient having delicate skin.

In order to achieve the above object, according to the present invention, there is provided a pulse photometry probe, comprising:

a tape-shaped compressive bandage section, having a first surface, a second surface opposing to the first surface, a first through hole and a second through hole each connecting the first surface and the second surface, the bandage section is adapted to be wound around a living tissue such that the first surface is brought into contact with the living tissue;

a light emitter, which emits light toward the living tissue, the light emitter detachably attached on a first position of the second surface such that the light passes through the first through hole; and

a light receiver, which receives the light which has been emitted from the light emitter and transmitted through the living tissue, the light receiver detachably attached on a second position of the second surface such that the light passes through the second through hole.

In such a configuration, the surface of the bandage section to be brought into contact with a living tissue has no adhesive layer. Hence, a decrease in adhesion of the bandage section, which would otherwise be caused when a probe is removably attached to and brought into contact with a living tissue numerous times, does not arise. The light emitter and the light receiver are not brought into direct contact with living tissue. Further, since the bandage section is compressive, local pressure exerted on living tissue can be prevented and dispersed when the probe is attached to the living tissue.

Preferably, the bandage section includes: a compressive member, having a first surface which constitutes a first section of the first surface of the bandage section, and a second surface opposing to the first surface; and a cover tape, having an adhesive surface adhered on the second surface of the compressive member while constituting a second section of the first surface of the bandage section, and a non-adhesive surface opposing to the adhesive surface so as to constitute the second surface of the bandage section. The second section of the first surface of the bandage section serves as a fastener which maintains a state in which the bandage section is wound around the living tissue.

In such a configuration, by a cover tape of a single member, the compressible member can be supported by an adhesive surface to be bonded to the compressible member, and the wound state of the bandage section can be maintained.

Here, it is preferable that at least one of the compressive member and the cover tape is made non-transparent.

In such a configuration, external light is prevented by the compressible member or the cover tape, thereby improving the accuracy of measurement.

Further, it is preferable that the compressive member is comprised of a sponge.

In such a configuration, since a sponge comes into contact with the living tissue, no decrease arises in adhesion of the probe, which would otherwise be caused when the probe is removably attached to living tissue numerous times.

Further, by compressibility of the sponge, there can be prevented exertion of local pressure onto living tissue, which would otherwise arise when the probe is attached to the subject.

Preferably, the bandage section includes a first plate member and a second plate member which respectively disperse a pressure from the light emitter and the light receiver to the living tissue, generated when the bandage section is wound around the living tissue.

In such a configuration, local pressure exerted on the living tissue from the light emitter and the light receiver, which would otherwise be caused by wrapping and attachment of the probe, is dispersed, thereby preventing damage to living tissue, such as necrosis.

Here, it is preferable that the first plate member is a non-transparent member having a first through hole which forms a part of the first through hole, and the second plate member is a non-transparent member having a second through hole which forms a part of the second through hole.

In such a configuration, external light is prevented by the plate members, thereby improving the accuracy of measurement.

Preferably, a first adhesive member is provided on the first position, and a second adhesive member is provided on the second position.

In such a configuration, the ease of operation for attaching the light emitter and the light receiver to the bandage section becomes superior. Use of the bandage section as a disposable member and reuse of the light emitter and light receivers enable cost-effective use of a probe.

Here, it is preferable that the first adhesive member is a non-transparent member having a through hole which forms a part of the first through hole, and the second adhesive member is a non-transparent member having a through hole which forms a part of the second through hole.

In such a configuration, external light can be shielded, so that the accuracy of measurement can be improved. Through holes are provided for permitting passage of the light, thereby enabling emission and reception of only required light.

Further, it is preferable that either one of a hook portion and a loop portion of a hook-and-loop fastener is provided at the first position and the second position. The other one of the hook portion and the loop portion of the hook-and-loop fastener is provided on each of the light emitter and the light receiver.

In such a configuration, removal and attachment of the light emitter and the light receiver to the bandage section are performed by way of a hook-and-loop fastener. Hence, even when removal and attachment operations are performed repeatedly, the operations are certainly performed .

Here, it is preferable that each of the hook portion and the loop portion is a non-transparent member having a through hole which forms a part of either one of the first through hole and the second through hole.

In such a configuration, external light can be shielded, so that the accuracy of measurement can be improved. Through holes are provided for permitting passage of the light, thereby enabling emission and reception of only required light.

Still further, it is preferable that the first adhesive member and the second adhesive member are elongated such that both elongating axes are angled by 60 to 120 degrees.

It is also preferable that the hook-and-loop fastener at the first position and the hook-and-loop fastener at the second position are elongated such that both elongating axes are angled by 60 to 120 degrees.

Alternatively, it is preferable that a proximal end of a first lead wire connected to the light emitter and a proximal end of a second lead wire connected to the light receiver are angled by 60 to 120 degrees.

In such configurations, when the probe is attached to the subject, lead wires of the light emitter and the light receiver can be readily withdrawn.

Still further, it is preferable that each of the light emitter and the light receiver at least partly has a pile fabric surface to be attached to the bandage section.

In such a configuration, the light emitter and the light receiver can be readily detached from the bandage section.

Preferably, the pulse photometry probe further comprises at least one transparent sheet member, attached on the first surface of the bandage member so as to cover the first through hole and the second through hole.

In such a configuration, it is prevented risk of necrosis, which could otherwise be caused when excessive pressure is exerted on the portion of the living tissue with which the edges of the through holes formed in the bandage section come into contact when the probe is attached to the living tissue.

According to the present invention, there is also provided a pulse photometry probe, comprising:

a tape-shaped compressive bandage section, having a first surface, a second surface opposing to the first surface, the bandage section is adapted to be wound around a living tissue such that the first surface is brought into contact with the living tissue;

a light emitter, which emits light toward the living tissue, the light emitter detachably attached on a first position of the second surface; and

a light receiver, which receives the light which has been emitted from the light emitter and transmitted through the living tissue, the light receiver detachably attached on a second position of the second surface,

wherein a thickness of the bandage section at the first position and the second position is determined so as to allow the light to pass through.

Preferably, each of the light emitter and the light receiver at least partly has a pile fabric surface to be attached to the bandage section.

According to the present invention, there is also provided a method of applying a pulse photometry probe to a living tissue, comprising the steps of:

attaching a light emitter and a light receiver onto a bandage section such that a first proximal end of a first lead wire connected to the light emitter and a second proximal end of a second lead wire connected to the light receiver are angled by a predetermined angle; and

wounding the bandage section around the living tissue such that an optical axis of the light emitter coincides with an optical axis of the light receiver.

Preferably, the predetermined angle is in a range of 60 to 120 degrees, and the living tissue is a foot of a human body.

### BRIEF DESCRIPTION OF THE DRAWINGS

The above objects and advantages of the present invention will become more apparent by describing in detail preferred exemplary embodiments thereof with reference to the accompanying drawings, wherein:

FIG. 1 is a view showing the appearance of a pulse photometry probe according to a first embodiment of the invention;

FIG. 2 is an exploded view of a bandage section of the pulse photometry probe of the first embodiment;

FIG. 3 is a cross-sectional view of the bandage section of the pulse photometry probe of the first embodiment;

FIG. 4 is a view showing the manner in which the pulse photometry probe of the first embodiment is attached to a finger;

FIGS. 5A and 5B are views showing the manner in which the pulse photometry probe of the first embodiment is attached to a foot;

FIG. 6 is an exploded perspective view of a pulse photometry probe according to a second embodiment of the invention;

FIG. 7 is a cross-sectional view of a light emitter or light receiver of the pulse photometry probe of the second embodiment;

FIG. 8 is a perspective view of a light emitter or light receiver of the pulse photometry probe of the second embodiment;

FIG. 9 is an exploded view of a pulse photometry probe according to a third embodiment of the invention;

FIG. 10 is a view showing the manner in which the pulse photometry probe of the third embodiment is attached to a foot;

FIG. 11 is an exploded perspective view showing a pulse photometry probe according to a fourth embodiment of the invention;

FIG. 12 is a cross-sectional view of the pulse photometry probe of the fourth embodiment;

FIG. 13 is an exploded perspective view a modified example of the pulse photometry probe of the fourth embodiment;

FIG. 14 is an exploded perspective view showing a pulse photometry probe according to a fifth embodiment of the invention; and

FIG. 15 is a cross-sectional view of the pulse photometry probe of the fifth embodiment.

### DETAILED DESCRIPTION OF THE PREFERRED EMBODIMENTS

Preferred embodiments of a pulse photometry probe according to the invention will be described in detail hereinbelow with reference to the accompanying drawings.

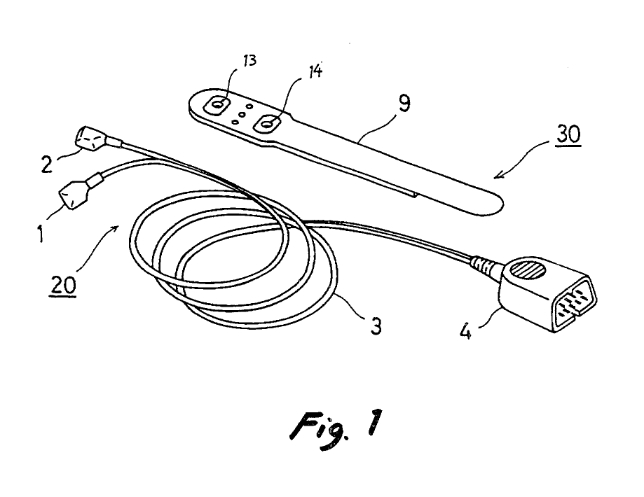

FIG. 1 shows the appearance of a pulse photometry probe according to a first embodiment of the invention. The pulse photometry probe is roughly divided into an instrument section 20 and a bandage section 30.

The instrument section 20 comprises a light emitter 1 (an LED or the like) for emitting light; a light receiver 2 (a photodiode or the like) for receiving the light that has been emitted from the light emitter 1 and passed through living tissue; a Y-shaped cable 3 which transmits a light-emission drive signal to the light emitter 1 and transmits a received light signal from a light receiver 2 to a main unit (not shown); and a connector 4 to be attached to the main unit. The Y-shaped cable 3is constituted of two lead wires; one connected to the light emitter 1, and the other connected to the light receiver 2.

The light emitter 1 and the light receiver 2 are taken as individual measurement elements, thereby rendering a cable diameter small and the cable flexible. The distance between the light emitter 1 and the light receiver 2 is not fixed.

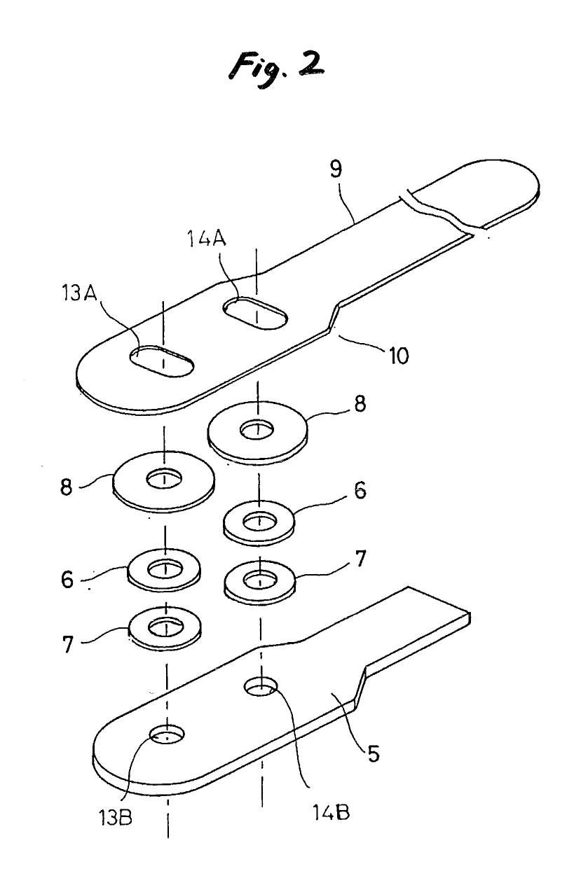

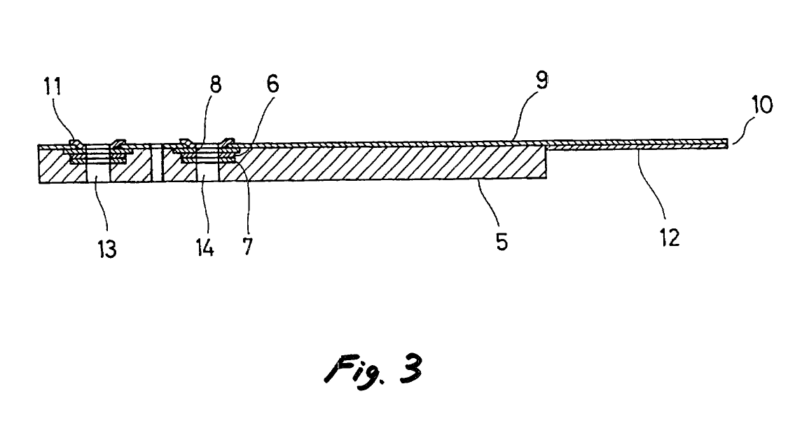

The bandage section 30 will now be described by reference to FIGS. 2 and 3. The bandage section 30 has a compressive sponge 5 which is to be wrapped directly around living tissue (e.g., a finger or a foot), and a cover tape 9 one side of which is an adhesive surface 10 to be attached to the sponge 5. The adhesive surface 10 of the cover tape 9 has a surface to be bonded to the sponge 5, and another surface not to be bonded to the sponge 5. When the bandage section 30 is wrapped around living tissue, the surface that is not to be brought into contact with the sponge 5 serves as a fastener for maintaining an attached state of the bandage section 30. Before the bandage section 30 is attached to living tissue, the adhesive surface that is not brought into contact with the sponge 5 is covered with backing paper 12.

A hook-and-loop fastener or the like may be used in place of the adhesive surface of a cover tape 9 which is not attached to the sponge 5.

The cover tape 9 may be made of an extendable bandage or a non-extendable paper tape. Preferably, the cover tape 9 is made of a nonwoven extendable tape over which adhesive is applied.

Since the only member to be brought into direct contact with living tissue is the sponge 5, the sponge 5 can softly contact irregularities in the surface of living tissue, thereby achieving superior adhesion and dispersion of pressure applied on the living tissue.

Further, the bandage section 30 has a first hole 13 for permitting passage of the light originating from the light emitter 1, and a second hole 14 for causing the light receiver 2 to receive the light that has been emitted from the light emitter 1 and has passed through the living tissue.

The light emitter 1 is removably attached to an upper surface of the cover tape 9 so as to be able to emit light by way of the first hole 13. The light receiver 2 is removably attached to the upper surface of the cover tape 9 so as to be able to receive light by way of the second hole 14. Black (non-transparent) annular double-sided tapes 8, each having a hole substantially identical in size with the first and second holes 13, 14, are removably attached to the respective first and second holes in a concentric manner.

A hole 13A constituting the first hole 13 and a hole 14A constituting the second hole 14, the first and second holes 13 and 14 being formed in the cover tape 9, are formed so as to become larger than a hole 13B constituting the first hole 13 and a hole 14B constituting the second hole 14, the first and second holes 13 and 14 being formed in the sponge 5. The light emitter 1 and the light receiver 2 are attached and bonded to the adhesive surface of the double-sided tape 8 that is exposed by way of the holes 13A and 14A.

Before the light emitter 1 and the light receiver 2 are bonded to the bandage section 30, areas of the adhesive surface of the tape 8, which are exposed by way of the holes 13A, 14A, are covered with backing paper 11. When the light emitter 1 and the light receiver 2 are attached to the bandage section 30, the backing paper 11 is peeled, whereby the light emitter 1 and the light receiver 2 are attached to the double-sided tape 8.

With such a structure, measurement elements, such as the light emitter 1 and the light receiver 2, are fixed to areas on the back of the bandage section 30 (i.e., on another surface of the tape section 30 opposite to a surface to be brought into contact with living tissue).

Moreover, the distance between the light emitter 1 and the light receiver 2 is not fixed. The first hole 13 or the second hole 14 is formed into an oval shape, whereby the distance between the light emitter 1 and the light receiver 2 can be adjusted at the time of attachment of the light emitter 1 and the light receiver 2 to the bandage section 30. As a result, the distance can be finely adjusted such that the optical axis of the light emitter 1 is aligned with that of the light receiver 2 at the time of attachment of the probe. Alternatively, a plurality of bandage sections 30, which differ from each other in terms of a distance between the first hole 13 and the second hole 14, are prepared in advance. In accordance with the size of a patient's living tissue (e.g., a finger), an appropriate bandage section 30 may be selected.

In order to distribute the pressure which is exerted on living tissue from the light emitter 1 and the light receiver 2 when the bandage section 30 is wrapped around the living tissue, plates 6 are provided concentrically with the first and second holes 13, 14 and at positions closer to the living tissue than the double-sided tape 8. In the embodiment, the plates 6 assume an annular shape and have the same size holes as that of the first and second holes 13, 14. Here, the plates 6 may assume the shape of a disk, so long as the plates are transparent. A surface of each plate 6 facing a living tissue is provided with the double-sided tape 7 and is to be attached to the sponge 5.

As a result of the plate 6 being provided with a surface larger than the attachment surface of the light emitter 1 and that of the light receiver 2, the plates 6 can distribute the pressure exerted on the surface of a living tissue (skin) in cooperation with a buffering action of the sponge 5. In particular, local pressure, which would arise in the edge of the light emitter 1 and that of the light receiver 2, can be prevented by the plates 6 and the sponge 5. The plates 6 may be made of ABS, PET, or vinyl chloride.

Although in the embodiment a double-sided tape is used for bonding individual components, heat welding may also be employed.

Although a black, non-transparent adhesive tape is used for the annular double-sided adhesive tapes 8, any one of the cover tape 9, the annular double-sided adhesive tapes 8, the annular plates 6, and the sponge 5 may be non-transparent, so that external light attributable to an error in measurement can be shielded.

As shown in FIG. 1, according to the embodiment, three holes are provided between the first hole 13 and the second hole 14 of the bandage section 30 with respect to the widthwise direction of the bandage section 30. These holes are provided for making the bandage section 30 flexible at the time of attachment of the bandage section 30 to a patient.

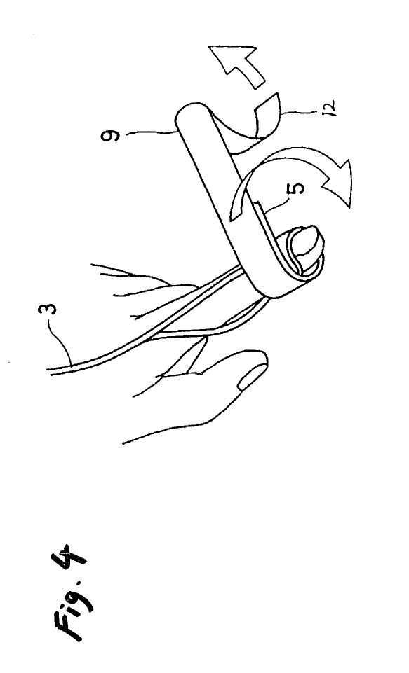

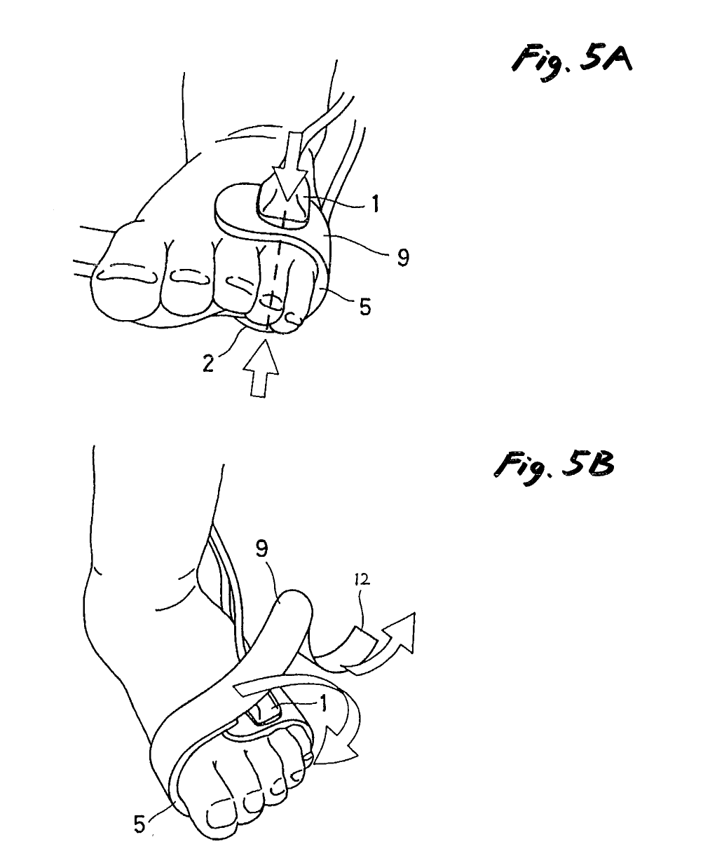

Attachment of the probe will now be described. The light emitter 1 and the light receiver 2 are fixed to measurement element attaching sections of the bandage section 30. The light emitter 1 and the light receiver 2 are oriented so as to oppose each other with a region of a patient to be measured (e.g., a finger or foot) (hereinafter simply called a “measurement region”) interposed therebetween. FIG. 4 shows the manner in which the bandage section 30 is attached to a finger while the backing paper 12 is being peeled. FIG. 5A shows the manner in which the light emitter 1 is attached to an instep and the light receiver 2 is attached to a sole.

The adhesive surface 10, which is located at the end of the bandage section 30 and serves as a fastener, is attached to the back of the wrapped cover tape 9, thereby placing and fixing the entire probe on the measurement region. FIG. 5B is a view showing the manner in which an attempt is made to attach the probe to a foot.

The strength required for wrapping is adjusted in accordance with living tissue (e.g., the color of the skin) located at a position distant from a measurement region or with the waveform of a measured pulse wave detected by the probe.

The light emitter 1 and the light receiver 2 are fixed to the measurement element attaching sections. However, if the bandage section 30 to be wrapped has a length sufficient for covering the back of the light emitter 1 and that of the light receiver 2, removal of the light emitter 1 and the light receiver 2 can be prevented.

If the bandage section 30 is used as a disposable part and the instrument section 20 is recycled, cost-efficient use of the probe becomes feasible.

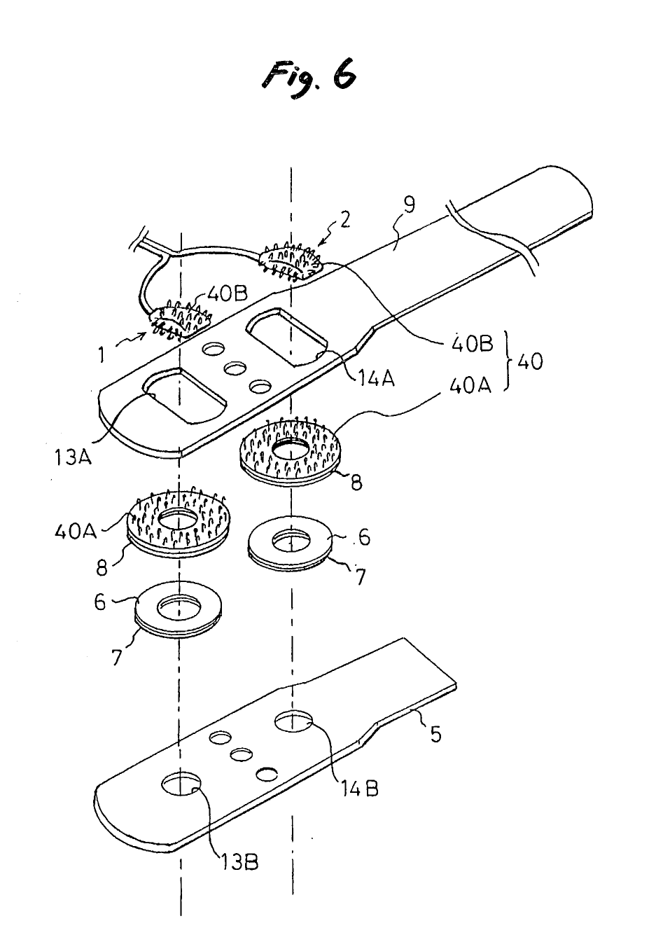

FIG. 6 shows a second embodiment of the invention. In the embodiment, a hook-and-loop fastener 40 is used for the probe of the first embodiment in order to attach the light emitter 1 and the light receiver 2 to the bandage section 30. In other words, a hook section 40A of the hook-and-loop fastener 40 is attached to the surface of double-sided adhesive tape 8 facing the cover tape 9. A loop section 40B of the hook-and-loop fastener 40 is attached to the respective surface of the light emitter 1 and that of the light receiver 2.

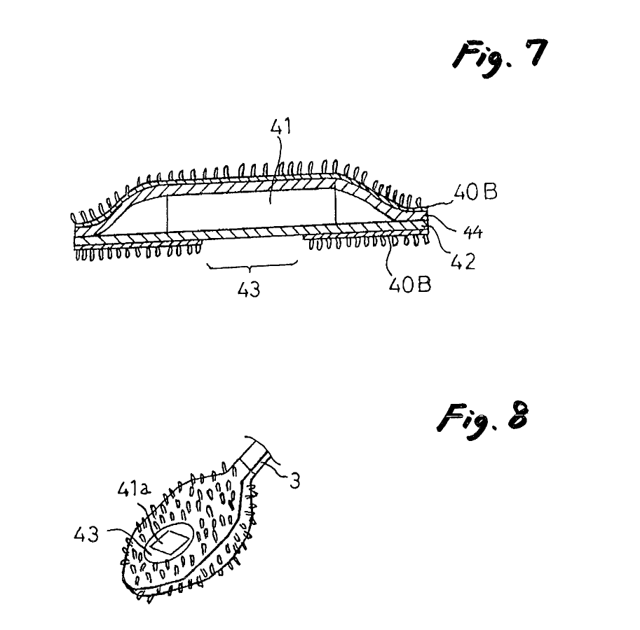

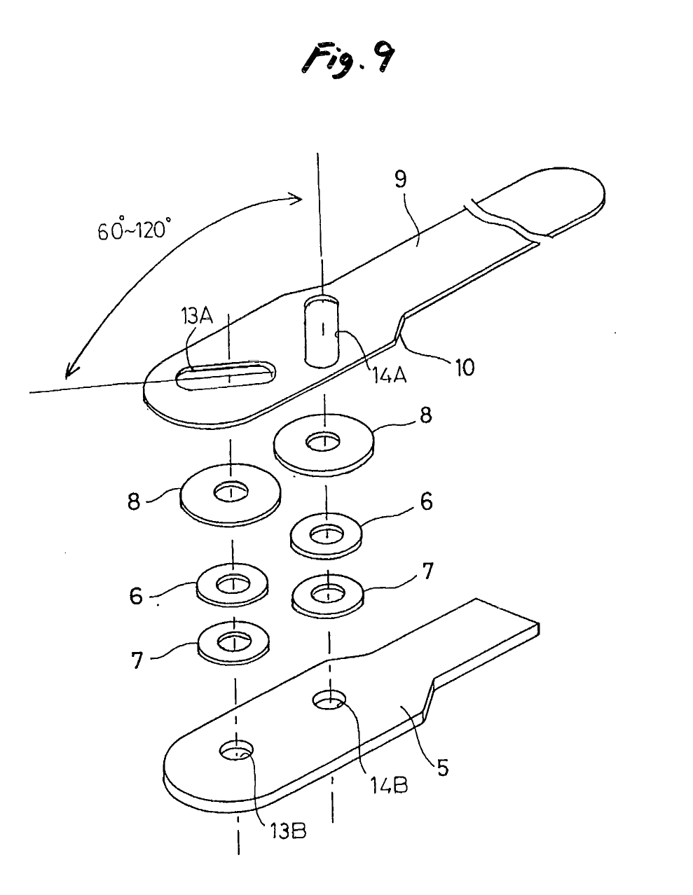

The configuration of the light emitter 1 or that of the light receiver 2 is shown in the cross-sectional view of FIG. 7. An appearance of the light emitter 1 or that of the light receiver 2 is shown in FIG. 8. As illustrated, a light-emitting element or light-receiving element 41 is covered with two sheets; namely, a transparent sheet 42 provided on a light-emitting or a light-receiving surface of the element, and a light-shielding sheet 44 provided on the other side of the element. For instance, a vinyl chloride sheet is used for these sheets 42, 44, and peripheries of the sheets are bonded together by high-frequency welding. The loop section 40B of the hook-and-loop fastener 40 is attached to the entire exterior surfaces of the two sheets 42, 44. A window 43 is formed in a light-emitting surface or light-receiving surface 41a in the loop section 40B provided on the light-emitting or light-receiving surface of the light-emitting element or light-receiving element 41 so as not to shield light. In other respects, the probe is identical in structure with that described in the previous embodiment.

Since the probe employs the hook-and-loop fastener 40, even when the light emitter 1 and the light receiver 2 are repeatedly attached to and removed from the bandage section 30, removal and attachment of the light emitter 1 and the light receiver 2 can be performed repeatedly.

In the embodiment, the loop section 40B is provided on the surface of the light emitter 1 and that of the light receiver 2. The hook section 40A is provided on the bandage section 30. The hook section 40A and the loop section 40B may be reversed. However, since the loop section 40B provides a more comfortable feeling, for an operator who often touches the light emitter 1 and the light receiver 2 it is preferable to provide the light emitter 1 and the light emitter 2 with the loop section 40B.

FIG. 9 shows a third embodiment of the invention. The embodiment is analogous to the probe described in connection with the first embodiment, and holes 13A and 14A formed in the cover tape 9 are angled by 60° to 120° with respect to the major axes of the holes. Adhesive surface portions of the double-sided tape 8 exposed through the cover tape 9 are formed and elongated such that a proximal end of a lead wire of the light emitter 1 and a proximal end of a lead wire of the light receiver 2 are spaced apart from each other and angled by 60° to 120° when the bandage section 30 is placed in a horizontal state and the light emitter and the light receiver are attached to the double-sided tape 8 in a predetermined condition. In other respects, the probe is identical in structure with that of the first embodiment.

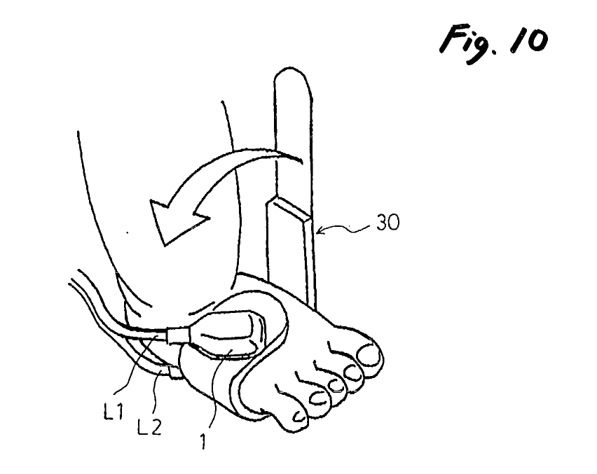

By such a configuration, when the probe is attached to a patient and the light emitter 1 and the light receiver 2 are attached in the strongest manner to the adhesive surface portions of the double-sided tape 8 exposed through the holes 13A and 14A; that is, when the surface to be bonded becomes maximum, a proximal end L1 of the lead wire of the light emitter 1 and a proximal end L2 of the lead wire of the light receiver 2 can be easily withdrawn, as shown in FIG. 10 showing the manner in which the probe is attached to a patient's foot. Thus, an operation for attaching the probe becomes easy.

The third embodiment is based on the first embodiment, and the holes 13A, 14A formed in the cover tape 9 are elongated such that major axes of the holes are angled by 60° to 120°. In the second embodiment, the holes 13A and 14A formed in the cover 9 may be elongated such that major axes of the holes are angled by 60° to 120°. In this case, the probe provides an easy withdrawal of lead wires and leads to a removable attachment of the light emitter 1 and the light receiver 2 repeatedly.

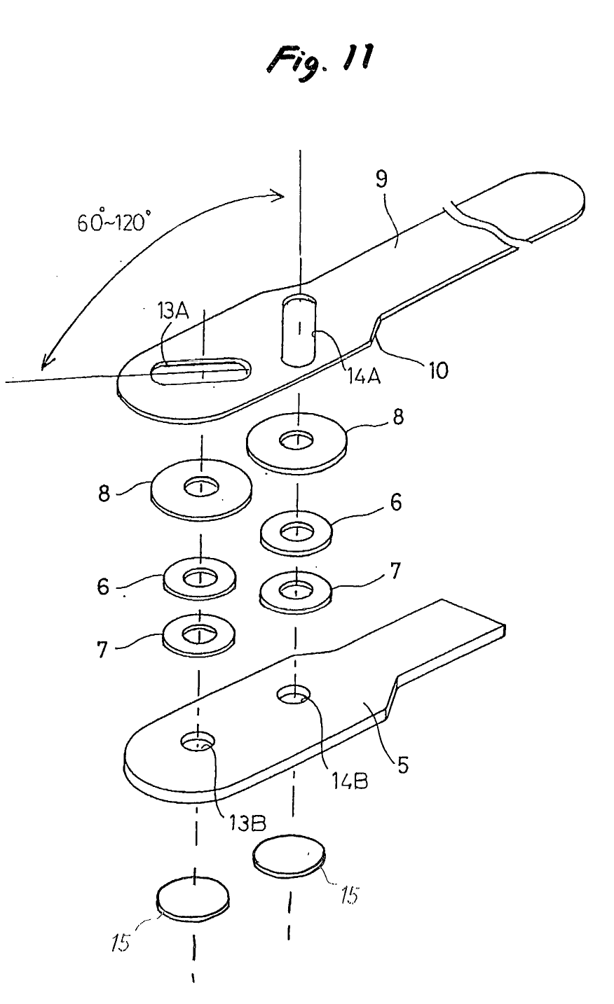

FIG. 11 is a view of a pulse photometry probe according to a fourth embodiment of the invention. The embodiment is based on the third embodiment, and holes 13B, 14B are sealed with two transparent sheets 15 (surgical tape or film is preferable) from the side surface of the sponge 5 which is to be brought into contact with living tissue.

The reason for sealing the transparent sheets 15 is to prevent risk of necrosis, which could otherwise be caused when excessive pressure is exerted on the portion of the living tissue with which the edges of the holes 13B, 14B formed in the sponge 5 come into contact when the probe is attached to the living tissue.

An appropriate example material for the transparent sheet 15 is polyurethane elastomer of about 0.3 to 0.4 mm thickness. Preferably, an adhesive is thinly applied to one surface of each of the transparent sheets 15, thereby sealing the sponge 5.

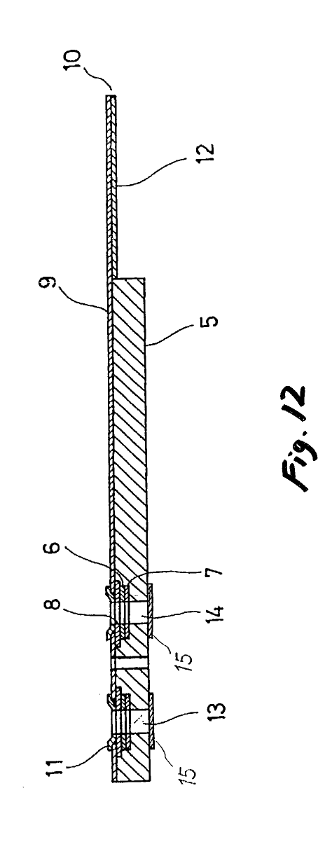

As shown in FIG. 12 which is a cross-sectional view of the pulse photometry probe, the holes 13, 14 are sealed with the transparent sheets 15.

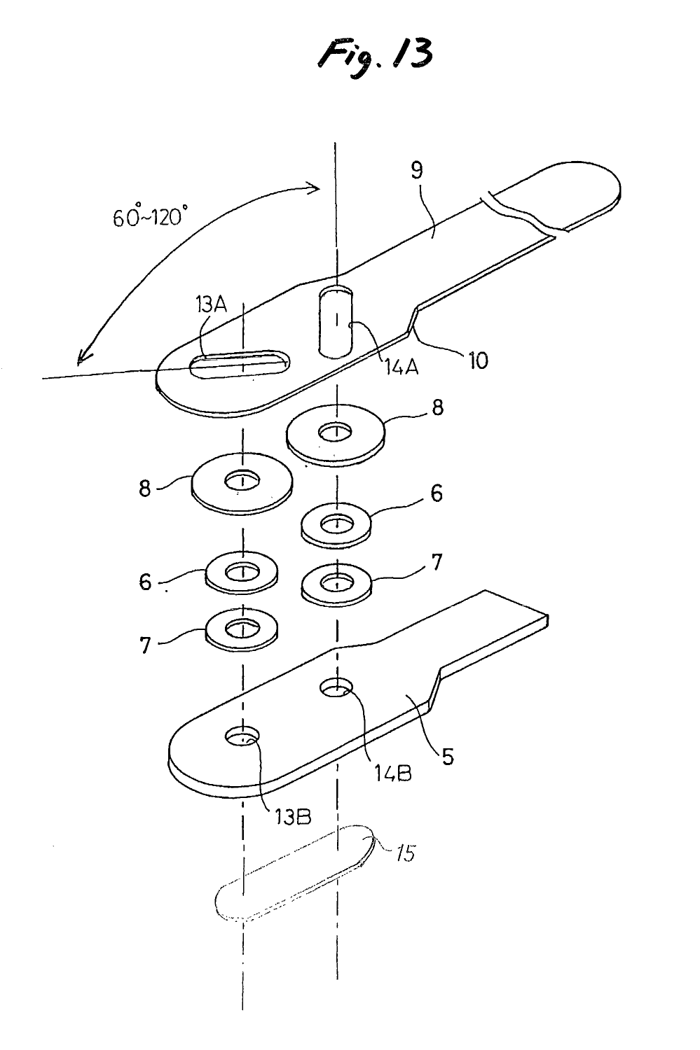

The transparent sheets 15 are not necessarily divided into two pieces. As shown in FIG. 13, the transparent sheet may be a single sheet of sufficient size for collectively sealing the holes 13B, 14B formed in the sponge 15.

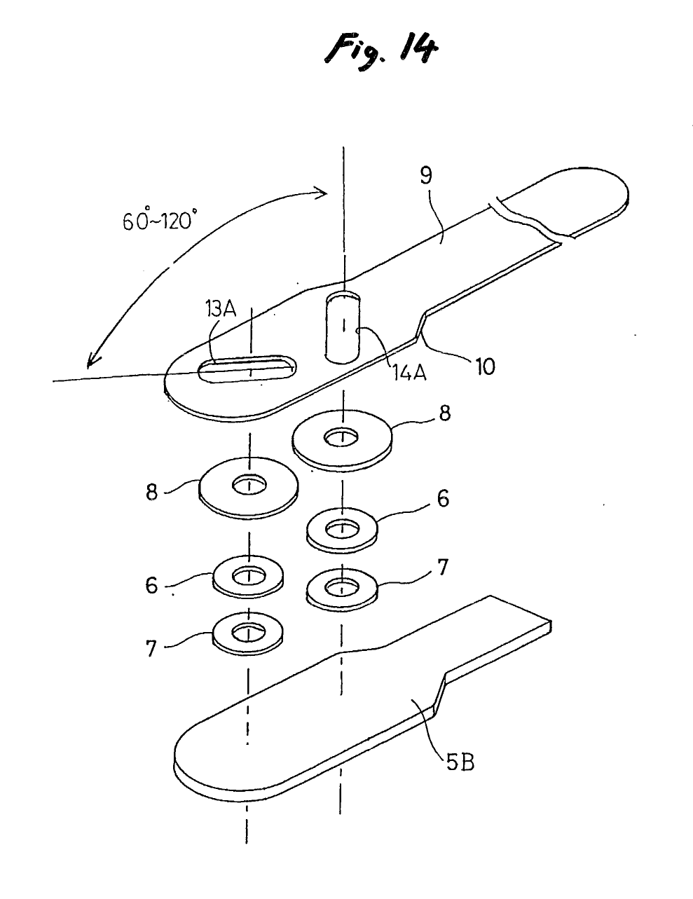

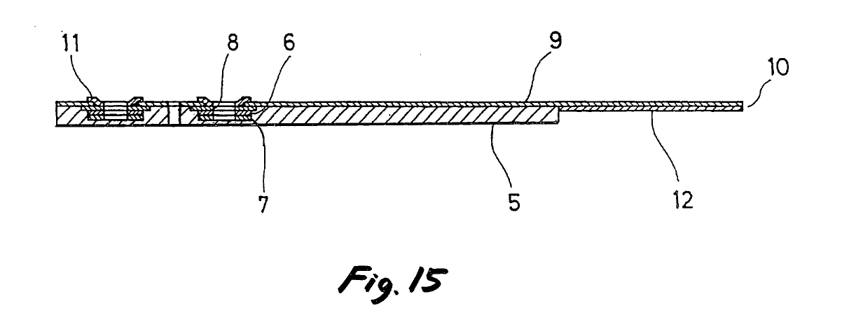

FIG. 14 is a view showing a pulse photometry probe according to a fifth embodiment of the invention. The embodiment is based on the third embodiment and characterized in that the sponge 5 is provided thinly instead of forming the holes 13B, 14B, to thereby impart to the probe a light transmission characteristic attributable to the porosity of the sponge 5. The purpose is to avoid forming edges of the holes of the sponge 5, as well as the fourth embodiment.

An appropriate example material of the sponge 5 is soft urethane foam. Sufficient porosity for imparting a light transmission characteristic to the sponge is about 50 to 60 cells in an area of 25 mm².

A preferable thickness of at least a portion of the sponge 5 through which light transmits is about 1 to 2 mm. The thickness of the other portions of the sponge 5 may be the same value; that is, 1 to 2 mm or thereabouts or a greater value. As shown in FIG. 15, light-passing portions of the sponge 5 are made thinner.

By such a configuration, when a probe is attached to living tissue and measurement of the living tissue is performed, the light originating from the light emitter 1 transmits through the living tissue and reaches the light receiver 2 through at least portions of the sponge 5 having a light-transmission property.

In the first through fifth embodiments, at least surface portions of the light emitter 1 and that of the light receiver 2, which come into contact with the cover tape 9, are made of a pile fabric, such as that of a hook-and-loop fastener, except for portions of the light emitter 1 and the light receiver 2 through which light transmits. Further, it may be the case that the surface of the double-sided adhesive tape 8 facing the cover tape 9 is not provided with a hook-and-loop fastener. In this case, the peel strength required for removing the light emitter 1 and the light receiver 2 from the portions of the tape 8 exposed through the cover tape 9 is weaker than that required when the light emitter 1, the light receiver 2, and the double-sided adhesive tape 8 are made of a hook-and-loop fastener. Therefore, the light emitter 1 and the light receiver 2 can be peeled from the cover tape 9.

Although the present invention has been shown and described with reference to specific preferred embodiments, various changes and modifications will be apparent to those skilled in the art from the teachings herein. Such changes and modifications are obvious to be deemed to come within the spirit, scope and contemplation of the invention as defined in the appended claims.

What is claimed is:

1. A pulse photometry probe, comprising:
a tape-shaped compressive bandage section, having a first surface, a second surface opposing to the first surface, a first through hole and a second through hole each connecting the first surface and the second surface, the bandage section is adapted to be wound around a living tissue such that the first surface is brought into contact with the living tissue;
a light emitter, which emits light toward the living tissue, the light emitter detachably attached on a first position of the second surface such that the light passes through the first through hole; and
a light receiver, which receives the light which has been emitted from the light emitter and transmitted through the living tissue, the light receiver detachably attached on a second position of the second surface such that the light passes through the second through hole.

2. The pulse photometry probe as set forth in claim 1, wherein:
the bandage section includes:
a compressive member, having a first surface which constitutes a first section of the first surface of the bandage section, and a second surface opposing to the first surface; and
a cover tape, having an adhesive surface adhered on the second surface of the compressive member while constituting a second section of the first surface of the bandage section, and a non-adhesive surface opposing to the adhesive surface so as to constitute the second surface of the bandage section; and
the second section of the first surface of the bandage section serves as a fastener which maintains a state in which the bandage section is wound around the living tissue.

3. The pulse photometry probe as set forth in claim 1, wherein the bandage section includes a first plate member and a second plate member which respectively disperse a pressure to the living tissue from the light emitter and the light receiver generated when the bandage section is wound around the living tissue.

4. The pulse photometry probe as set forth in claim 1, wherein a first adhesive member is provided on the first position, and a second adhesive member is provided on the second position.

5. The pulse photometry probe as set forth in claim 2, wherein at least one of the compressive member and the cover tape is made non-transparent.

6. The pulse photometry probe as set forth in claim 3, wherein the first plate member is a non-transparent member having a first through hole which forms a part of the first through hole, and the second plate member is a non-transparent member having a second through hole which forms a part of the second through hole.

7. The pulse photometry probe as set forth in claim 4, wherein the first adhesive member is a non-transparent member having a through hole which forms a part of the first through hole, and the second adhesive member is a non-transparent member having a through hole which forms a part of the second through hole.

8. The pulse photometry probe as set forth in claim 1, wherein:
either one of a hook portion and a loop portion of a hook-and-loop fastener is provided at the first position and the second position; and
the other one of the hook portion and the loop portion of the hook-and-loop fastener is provided on each of the light emitter and the light receiver.

9. The pulse photometry probe as set forth in claim 8, wherein each of the hook portion and the loop portion is a non-transparent member having a through hole which forms a part of either one of the first through hole and the second through hole.

10. The pulse photometry probe as set forth in claim 4, wherein the first adhesive member and the second adhesive member are elongated such that both elongating axes are angled by 60 to 120 degrees.

11. The pulse photometry probe as set forth in claim 8, wherein the hook-and-loop fastener at the first position and the hook-and-loop fastener at the second position are elongated such that both elongating axes are angled by 60 to 120 degrees.

12. The pulse photometry probe as set forth in claim 2, wherein the compressive member is comprised of a sponge.

13. The pulse photometry probe as set forth in claim 1, further comprising at least one transparent sheet member, attached on the first surface of the bandage member so as to cover the first through hole and the second through hole.

14. The pulse photometry probe as set forth in claim 4, wherein each of the light emitter and the light receiver at least partly has a pile fabric surface to be attached to the bandage section.

15. The pulse photometry probe as set forth in claim 1, wherein a proximal end of a first lead wire connected to the light emitter and a proximal end of a second lead wire connected to the light receiver are angled by 60 to 120 degrees.

16. A pulse photometry probe, comprising:
a tape-shaped compressive bandage section, having a first surface, a second surface opposing to the first surface, the bandage section is adapted to be wound around a living tissue such that the first surface is brought into contact with the living tissue;
a light emitter, which emits light toward the living tissue, the light emitter detachably attached on a first position of the second surface; and
a light receiver, which receives the light which has been emitted from the light emitter and transmitted through the living tissue, the light receiver detachably attached on a second position of the second surface,
wherein a thickness of the bandage section at the first position and the second position is determined so as to allow the light to pass through.

17. The pulse photometry probe as set forth in claim 16, wherein each of the light emitter and the light receiver at least partly has a pile fabric surface to be attached to the bandage section.

18. A method of applying a pulse photometry probe to a living tissue, comprising the steps of:
attaching a light emitter and a light receiver onto a bandage section such that a first proximal end of a first lead wire connected to the light emitter and a second proximal end of a second lead wire connected to the light receiver are angled by a predetermined angle; and
wounding the bandage section around the living tissue such that an optical axis of the light emitter coincides with an optical axis of the light receiver.

19. The applying method as set forth in claim 18, wherein the predetermined angle is in a range of 60 to 120 degrees.

20. The applying method as set forth in claim 18, wherein the living tissue is a foot of a human body.
